SEQUENCE LISTING (6 sequences)

SEQ ID NO: 1 - DNA, 22 bp, synthetic construct
  source 1..22: mol_type = other DNA
ggcttggctctgctaacacgtt

SEQ ID NO: 2 - DNA, 22 bp, synthetic construct
  source 1..22: mol_type = other DNA
ggacgtttgtaatgtccgctcc

SEQ ID NO: 3 - DNA, 46 bp, synthetic construct
  source 1..46: mol_type = other DNA
cagccagccgcagcacgttcgctcataggagatatggtagagccgc

SEQ ID NO: 4 - DNA, 51 bp, synthetic construct
  source 1..51: mol_type = other DNA
gagagaatttgtaccacctcccaccgggcacatagcagtcctagggacagt

SEQ ID NO: 5 - DNA, 17 bp, synthetic construct
  source 1..17: mol_type = other DNA
ctgcatacgacgtgtct

SEQ ID NO: 6 - DNA, 20 bp, synthetic construct
  source 1..20: mol_type = other DNA
accatctatgactgtacgcc

(19) United States
(12) Patent Application Publication
(10) Pub. No.: US 2025/0334521 A1
(43) Pub. Date: Oct. 30, 2025

(54) CONFIRMING METHOD FOR DISPERSION CONDITION OF DIGITAL NUCLEIC ACID AMPLIFICATION REACTION SAMPLES IN REACTION AREAS

(71) Applicant: Caduceus Biotechnology Inc., Hsinchu County (TW)

(72) Inventors: Jung-Po Chen, Hsinchu County (TW); Yueh-Lun Ho, Hsinchu County (TW); Chien-Chung Pien, Hsinchu County (TW); Kuang Wu Hsu, Hsinchu County (TW); Jean-Dow Lee, Hsinchu County (TW); Rung-Ywan Tsai, Hsinchu County (TW)

(73) Assignee: Caduceus Biotechnology Inc., Hsinchu County (TW)

(21) Appl. No.: 18/679,388

(22) Filed: May 30, 2024

(30) Foreign Application Priority Data
Apr. 25, 2024 (TW) 113115350

(51) Int. Cl. G01N 21/64; C12Q 1/6806; C12Q 1/686; C12Q 1/6876

(52) U.S. Cl. CPC G01N 21/6486; C12Q 1/6806; C12Q 1/686; C12Q 1/6876

(57) ABSTRACT

A confirming method for dispersion conditions of digital nucleic acid amplification reaction samples in reaction areas includes the following steps. The nucleic acid reaction samples are dispersed in the reaction areas. The nucleic acid reaction samples include nucleic acid primers and fluorescent indicator molecules for nucleic acid amplification. Parts of the nucleic acid primers are combined with the fluorescent indicator molecules for nucleic acid amplification before a digital nucleic acid amplification reaction. Next, fluorescence is excited using an excitation light source with a first fluorescence excitation band, and fluorescence images are detected using a detector for detecting a first fluorescence emission band for the nucleic acid reaction samples dispersed in the reaction areas. Afterwards, whether the nucleic acid reaction samples are evenly distributed in the reaction areas is determined using the fluorescence images.

### CROSS-REFERENCE TO RELATED APPLICATION

This application claims the priority benefit of Taiwan application serial no. 113115350, filed on Apr. 25, 2024. The entirety of the above-mentioned patent application is hereby incorporated by reference herein and made a part of this specification.

### REFERENCE TO A SEQUENCE LISTING

The instant application contains a Sequencing Listing which has been submitted electronically in XML file and is hereby incorporated by reference in its entirety. Said XML copy, created on May 16, 2024, is named 143481-US-sequence listing and is 6,592 bytes in size.

### BACKGROUND

### Technical Field

The disclosure relates to a confirming method for dispersion conditions of digital nucleic acid amplification reaction samples in reaction areas.

### Description of Related Art

With the development of nucleic acid technology, various nucleic acid amplification detection technologies have been invented and applied to nucleic acid detection related to pathogens or cancer, such as a quantitative polymerase chain reaction (qPCR) and loop-mediated isothermal amplification (LAMP). In practical applications, the technologies must use measurement standards and make calibration curves to achieve the objective of nucleic acid quantification. In order to overcome the issue, the digital nucleic acid detection technology uses microfluidic technology to disperse (partition) nucleic acid samples in multiple microwells or multiple microdroplets. After nucleic acid amplification, if there are target nucleic acids in parts of the microwells or microdroplets, amplification signals (for example, fluorescent signals) can be detected in the parts of the microwells or the microdroplets. Through digitizing nucleic acid amplification and using a Poisson distribution, the objective of absolute quantification, such as digital PCR or digital LAMP detection, of nucleic acids can be achieved.

The digital nucleic acid detection technology disperses the nucleic acid samples into the microwells or the microdroplets through the microfluidic technology. In order to reduce detection errors, the microwells or the microdroplets with poor sample dispersion, such as (1) the microwells with bubbles therein, resulting in smaller volumes of the nucleic acid samples in the microwells or even no nucleic acid samples; and (2) some microdroplets with volumes being too large or too small when generated, resulting in larger or smaller volumes of the nucleic acid samples in the microdroplets, must be deducted. The prior art includes the following methods of (1) using additional addition of a non-nucleic acid amplification fluorescent indicator; (2) additionally adding a large amount of a specific nucleic acid and a corresponding primer, probe, and nucleic acid amplification fluorescent indicator; (3) using a scattering signal (U.S. Pat. No. 9,417,190 B2), etc. to try to solve the above issue. However, for the above methods, the additional non-nucleic acid amplification fluorescent indicator or the specific nucleic acid and the corresponding primer, probe, and fluorescent indicator for nucleic acid amplification must be added, and an excitation light source with another wavelength and a photography system for another fluorescence emission band must be added or a scattering signal detection system must be added. In this way, experimental methods may be cumbersome and subjected to additional burden, thereby affecting the convenience and efficiency of experiments.

Based on the above, developing a confirming method for dispersion conditions of digital nucleic acid amplification reaction samples in reaction areas to confirm whether the nucleic acid samples are effectively and evenly dispersed in multiple microwells or multiple microdroplets to further reduce cumbersome experimental methods and additional burden, thereby preventing affecting the convenience and efficiency of experiments, is an important topic currently needed.

### SUMMARY

The disclosure provides a confirming method for dispersion conditions of digital nucleic acid amplification reaction samples in reaction areas, which is used to confirm whether the nucleic acid samples are effectively and evenly dispersed in multiple microwells or multiple microdroplets to further reduce cumbersome experimental methods and additional burden, thereby preventing affecting the convenience and efficiency of experiments.

The disclosure provides a confirming method for dispersion conditions of digital nucleic acid amplification reaction samples in reaction areas, which includes the following steps. The nucleic acid reaction samples are dispersed in the reaction areas. The nucleic acid reaction samples include nucleic acid primers and fluorescent indicator molecules for nucleic acid amplification. Parts of the nucleic acid primers are combined with the fluorescent indicator molecules for nucleic acid amplification before a digital nucleic acid amplification reaction. Next, fluorescence is excited using an excitation light source with a first fluorescence excitation band, and fluorescence images are detected using a detector for detecting a first fluorescence emission band for the nucleic acid reaction samples dispersed in the reaction areas. Afterwards, whether the nucleic acid reaction samples are evenly distributed in the reaction areas is determined using the fluorescence images.

In an embodiment of the disclosure, the digital nucleic acid amplification reaction includes digital polymerase chain reaction (PCR) or digital loop-mediated isothermal amplification (LAMP).

In an embodiment of the invention, the reaction areas include multiple microwells or multiple microdroplets.

In an embodiment of the disclosure, a concentration of the primer is 0.2 μM to 1.6 μM, and a concentration of the fluorescent indicator molecules for nucleic acid amplification is 0.5 μM to 1 μM.

In an embodiment of the disclosure, the fluorescent indicator molecules for nucleic acid amplification include fluorescent dyes such as SYBR Green, EvaGreen, and SYTO.

In an embodiment of the disclosure, 5% to 95% of the nucleic acid primers are combined with the fluorescent indicator molecules for nucleic acid amplification.

In an embodiment of the disclosure, passband of the first fluorescence excitation band and a second fluorescence excitation band is from 446 nm to 486 nm; and passband of the first fluorescence emission band and a second fluorescence emission band is from 506 nm to 534 nm.

In an embodiment of the disclosure, a method for determining whether the nucleic acid reaction samples are evenly dispersed in the reaction areas using the fluorescence images includes analyzing and comparing fluorescence intensities of the fluorescence images.

Based on the above, the disclosure provides the confirming method for the dispersion conditions of the digital nucleic acid amplification reaction samples in the reaction areas, which may be used to confirm whether the nucleic acid samples are effectively and evenly dispersed in the microwells or the microdroplets, thereby reducing detection errors. In addition, in the disclosure, the fluorescence is excited using the excitation light source with the first fluorescence excitation band, and the fluorescence images are detected using the detector for detecting the first fluorescence emission band, and whether the samples are effectively and evenly dispersed in the reaction areas is then determined using the fluorescence images. The first fluorescence excitation band of the excitation light source and the first fluorescence emission band of the detector before the digital nucleic acid amplification reaction are the same as the second fluorescence excitation band of the excitation light source and the second fluorescence emission band detected by the detector after the digital nucleic acid amplification reaction. Therefore, compared with the prior art, cumbersome experimental methods and additional burden can be further reduced, thereby preventing affecting the convenience and efficiency of experiments.

### BRIEF DESCRIPTION OF THE DRAWINGS

FIG. 1 is a structural view of a fluorescence photography system according to an embodiment of the disclosure.

FIG. 2A and FIG. 2B are respectively fluorescence images of a sample 01 and a sample 02 taken using a fluorescence photography system before the sample 01 and the sample 02 are heated.

FIG. 3 is a fluorescence image of a microwell array chip, in which a LAMP reaction reagent sample 03 is poorly dispersed in some microwells due to surface defects or flow channel defects of the microwell array chip, taken using a fluorescence photography system before heating the microwell array chip.

FIG. 4 is a fluorescence image of a microwell array chip, in which a LAMP reaction reagent sample 03 is evenly dispersed, taken using a fluorescence photography system before heating the microwell array chip.

FIG. 5 is a partially enlarged view of FIG. 3.

FIG. 6 is a partial fluorescence image of a microwell array chip taken using a fluorescence photography system after heating the microwell array chip (the same region as FIGS. 5) to 65° C. for 45 minutes.

### DESCRIPTION OF THE EMBODIMENTS

Hereinafter, embodiments of the disclosure will be described in detail. However, the embodiments are illustrative, and the disclosure is not limited thereto.

The disclosure provides a confirming method for dispersion conditions of digital nucleic acid amplification reaction samples in reaction areas. In the following, the terms used in the specification are first defined and explained.

“Quantitative polymerase chain reaction (qPCR)” or “real-time quantitative PCR” refers to an experimental method that uses PCR to amplify and simultaneously quantify target DNA. Various assay chemistries are used for quantitation (including fluorescent dyes such as SYBR® green, fluorescent reporter oligonucleotide probes such as Taqman probes, etc.), and instant quantification is performed as the amplified DNA accumulates in the reaction after each amplification cycle.

“Loop-mediated isothermal amplification (LAMP)” is a constant-temperature loop nucleic acid amplification method. The LAMP reaction requires 3 sets of primer pairs, namely an outer primer pair, an inner primer pair, and a loop primer pair, wherein the primer pairs must be designed according to the target nucleic acid sequence, so that the detection of the target nucleic acid sequence is specific. In addition to the primer pairs, the LAMP reaction also needs to add Bst DNA polymerase. This polymerase may polymerize with the primer pairs at an optimal temperature (60-65° C.) to amplify DNA fragments with self-complementary ability and form a dumbbell-shaped structure with stem-loops, whereby the reaction is continuous and repeated to generate a large number of DNA fragments.

The terms “nucleic acid”, “nucleotide”, and “polynucleotide” are used interchangeably and refer to a polymer of DNA or RNA in a single-stranded or double-stranded form. Unless otherwise indicated, the terms encompass polynucleotides containing known analogs of natural nucleotides, and the polynucleotides have similar binding properties to the reference nucleic acid and are metabolized in a manner similar to naturally occurring nucleotides.

The term “primer” refers to an oligonucleotide. When the oligonucleotide is placed under conditions that induce the synthesis of a primer extension product, such as in the presence of a nucleotide and a polymerization inducer (for example, DNA or ribonucleic acid polymerase) and at a suitable temperature, pH, metal ion concentration, and salt concentration, the oligonucleotide is used to initiate the synthesis of complementary nucleic acid strands.

The disclosure provides a confirming method for dispersion conditions of digital nucleic acid amplification reaction samples in reaction areas, which includes the following steps. The nucleic acid reaction samples are dispersed in the reaction areas. The nucleic acid reaction samples include nucleic acid primers and fluorescent indicator molecules for nucleic acid amplification. Parts of the nucleic acid primers are combined with the fluorescent indicator molecules for nucleic acid amplification before a digital nucleic acid amplification reaction. Next, fluorescence is excited using an excitation light source with a first fluorescence excitation band, and fluorescence images are detected using a detector for detecting a first fluorescence emission band for the nucleic acid reaction samples dispersed in the reaction areas. Afterwards, whether the nucleic acid reaction samples are evenly distributed in the reaction areas is determined using the fluorescence images. The first fluorescence excitation band of the excitation light source and the first fluorescence emission band of the detector before the digital nucleic acid amplification reaction are the same as a second fluorescence excitation band of the excitation light source and a second fluorescence emission band detected by the detector after the digital nucleic acid amplification reaction. Passband of the first fluorescence excitation band and the second fluorescence excitation band is, for example, from 446 nm to 486 nm, and passband of the first fluorescence emission band and the second fluorescence emission band is, for example, from 506 nm to 534 nm.

In the embodiment, the digital nucleic acid amplification reaction may include digital PCR or LAMP, and the reaction areas may include multiple microwells or multiple microdroplets. The fluorescent indicator molecules for nucleic acid amplification may include fluorescent dyes such as SYBR Green, EvaGreen, and SYTO. A concentration of primer is, for example, 0.2 to 1.6 μM. A concentration of fluorescent indicator molecules for nucleic acid amplification is, for example, 0.5 to 1 μM. About 5% to 95% of the nucleic acid primers may be combined with the fluorescent indicator molecules for nucleic acid amplification. A method for determining whether the nucleic acid reaction samples are evenly dispersed in the reaction areas using the fluorescence images includes analyzing and comparing fluorescence intensities of the fluorescence images.

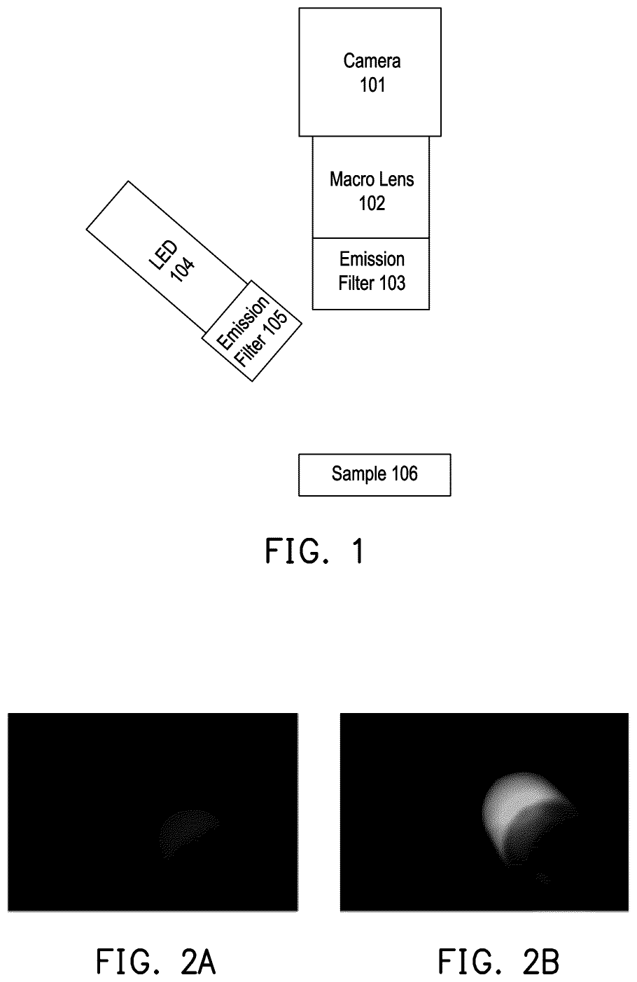

FIG. 1 is a structural view of a fluorescence photography system according to an embodiment of the disclosure. Please refer to FIG. 1. The fluorescence photography system of the disclosure may include a complementary metal oxide semiconductor (CMOS) camera 101, a macro lens 102, a fluorescence emission filter 103, a light emitting diode (LED) light source 104, and a fluorescence excitation filter 105. Fluorescence is excited using the LED light source, and fluorescence images are taken using the CMOS camera 101 for a sample 106. In the embodiment, the specification of the macro lens 102 is, for example, 60 mm, F 2.8, the specification of the fluorescence emission filter 103 is, for example, 520/28 nm, that is, passband of the fluorescence emission filter is, for example, from 506 nm to 534 nm, the specification of the LED light source 104 is, for example, 470 nm, and the specification of the fluorescence excitation filter 105 is, for example, 466/40 nm, that is, passband of the fluorescence excitation filter is, for example, from 446 nm to 486 nm.

Hereinafter, the confirming method for the dispersion conditions of the digital nucleic acid amplification reaction samples in the reaction areas of the disclosure will be explained in detail by experimental examples. However, the following experimental examples are not intended to limit the disclosure.

### EXPERIMENTAL EXAMPLE

### Example 1

### Prepare LAMP Reaction Reagent in Microcentrifuge Tube

According to Table 1 and Table 2, LAMP reaction reagent sample 01 and sample 02 were prepared in microcentrifuge tubes, wherein the LAMP reaction reagent sample 01 did not include primers, and the LAMP reaction reagent sample 02 included primers. Before heating the sample 01 and the sample 02, fluorescence images of the sample 01 and the sample 02 were taken using the fluorescence photography system of FIG. 1. The excitation light source was a 470 nm LED, and the exposure time was 100 ms. FIG. 2A and FIG. 2B are respectively fluorescence images of a sample 01 and a sample 02 taken using a fluorescence photography system before the sample 01 and the sample 02 are heated. The experimental results are shown in FIG. 2A and FIG. 2B, showing that exciting the LAMP reaction reagent sample 02 using the 470 nm LED (a fluorescence excitation filter: 466/40 nm) generated stronger green fluorescence (a fluorescence emission filter: 520/28 nm). Before a nucleic acid amplification reaction, parts of nucleic acid primers in the LAMP reaction reagent sample 02 were combined with fluorescent indicator molecules for nucleic acid amplification. The combined fluorescent indicator molecules for nucleic acid amplification were excited using the 470 nm excitation light source to generate fluorescence.

TABLE 1
LAMP reaction reagent formula

| | Sample 01 volume | Sample 02 volume |
|---|---|---|
| Reagent composition | (μl) | (μl) |
| 10x Isothermal Amplification | 2.15 | 2.5 |
| MgSO₄ (100 mM) | 1 | 1 |
| dNTP (10 mM) | 3.5 | 3.5 |
| λ F3 primer (10 mM) | 0 | 0.5 |
| λ B3 primer(10 mM) | 0 | 0.5 |
| λ FIP primer(10 mM) | 0 | 2 |
| λ BIP primer(10 mM) | 0 | 2 |
| λ loop F primer(10 mM) | 0 | 1 |
| λ loop B primer(10 mM) | 0 | 1 |
| 50x LAMP fluorescent dye | 0.5 | 0.5 |
| Bst 2.0 WarmStart DNA | 1 | 1 |
| Polymerase (8000 U/ml) | | |
| BSA (20 mg/ml) | 1.25 | 1.25 |
| Double-distilled water | 15.25 | 8.25 |
| (ddH₂O) | | |
| Total | 25 | 25 |

TABLE 2
LAMP primer sequence

| Name | Sequence (5′→3′) |
|---|---|
| λ F3 primer | GGCTTGGCTCTGCTAACACGTT |
| | (SEQ ID NO: 1) |
| λ B3 primer | GGACGTTTGTAATGTCCGCTCC |
| | (SEQ ID NO: 2) |
| λ FIP primer | CAGCCAGCCGCAGCACGTTCGC |
| | TCATAGGAGATATGGTAGAGCC |
| | GC (SEQ ID NO: 3) |
| λ BIP primer | GAGAGAATTTGTACCACCTCCC |
| | ACCGGGCACATAGCAGTCCTAG |
| | GGACAGT (SEQ ID NO: 4) |
| λ loop F primer | CTGCATACGACGTGTCT |
| | (SEQ ID NO: 5) |
| λ loop B primer | ACCATCTATGACTGTACGCC |
| | (SEQ ID NO: 6) |

### Example 2

### Digital LAMP of Microwell Array Chip

A microwell array chip was manufactured by injection molding and UV glue lamination, and the surface of the microwell array chip was made hydrophilic by coating or surface modification, wherein the diameter of microwell was 130 μm, the depth of microwell was 70 μm, and the pitch of microwells was 200 μm. There was a flow channel with a height of 300 μm, a width of 5 mm, and a length of 60 mm above the microwell array. Two ends of the flow channel were respectively provided with an injection hole and a ventilation hole. A LAMP reaction reagent sample 03 was prepared according to Table 3. 12 μL of the LAMP reaction reagent sample 03 and 80 μL of fluorinated liquid (FC40) were sequentially injected into the flow channel of the microwell array chip from the injection hole using a micropipette. During the process, the fluorinated liquid (FC40) pushed the LAMP reaction reagent sample 03 from the injection hole end toward the ventilation hole end, and the LAMP reaction reagent sample 03 was dispersed in multiple microwells and was isolated by the fluorinated liquid (FC40). Then, the microwell array chip was heated to 65° C. to perform a digital LAMP nucleic acid amplification reaction. During the dispersion process of the LAMP reaction reagent sample 03, the reaction reagent sample 03 may be poorly dispersed due to surface defects or flow channel defects of the microwell array chip (such as bubbles being partially generated or some microwells being filled with less LAMP reaction reagent sample 03), which causes quantitative bias in the digital LAMP nucleic acid amplification reaction. Therefore, the microwells with poor sample dispersion must be deducted before finding a Poisson distribution.

TABLE 3
LAMP reaction reagent sample 03 formula

| Reagent composition | Volume (μl) |
|---|---|
| λ DNA | 1 |
| 10x Isothermal Amplification | 2.5 |
| MgSO₄ (100 mM) | 1 |
| dNTP (10 mM) | 3.5 |
| λ F3 primer (10 mM) | 0.5 |
| λ B3 primer(10 mM) | 0.5 |
| λ FIP primer(10 mM) | 4 |
| λ BIP primer(10 mM) | 4 |
| λ loop F primer(10 mM) | 1 |
| λ loop B primer(10 mM) | 1 |
| 50x LAMP fluorescent dye | 0.5 |
| Bst 2.0 WarmStart DNA | 1 |
| Polymerase (8000 U/ml) | |
| BSA (20 mg/ml) | 1.25 |
| Double-distilled water (ddH₂O) | 3.25 |
| Total | 25 |

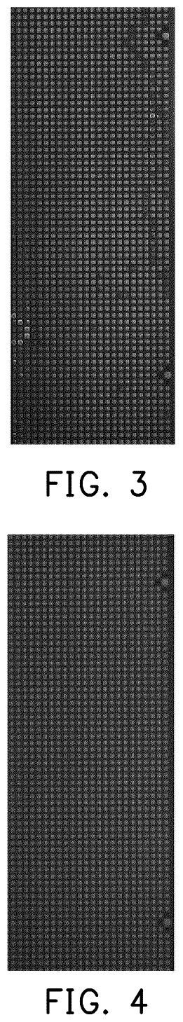

Before heating the microwell array chip, fluorescence images of the microwell array chip were taken using the fluorescence photography system of FIG. 1. The excitation light source was a 470 nm LED, and the exposure time was 560 ms. The experimental results are shown in FIG. 3 and FIG. 4, showing that exciting the LAMP reaction reagent sample 03 using the 470 nm LED (a fluorescence excitation filter: 466/40 nm) generated stronger green fluorescence (a fluorescence emission filter: 520/28 nm). Before a nucleic acid amplification reaction, parts of nucleic acid primers in the LAMP reaction reagent sample 03 were combined with fluorescent indicator molecules for nucleic acid amplification. The combined fluorescent indicator molecules for nucleic acid amplification were excited using the 470 nm excitation light source to generate fluorescence. In FIG. 3, the LAMP reaction reagent sample 03 was poorly dispersed in some microwells due to surface defects or flow channel defects of the microwell array chip. FIG. 4 shows that the LAMP reaction reagent sample 03 was evenly dispersed in the microwell array chip. Which microwells have bubbles or are filled with less LAMP reaction reagent sample 03 may be confirmed using the fluorescence images of the microwell array chip before the LAMP reaction.

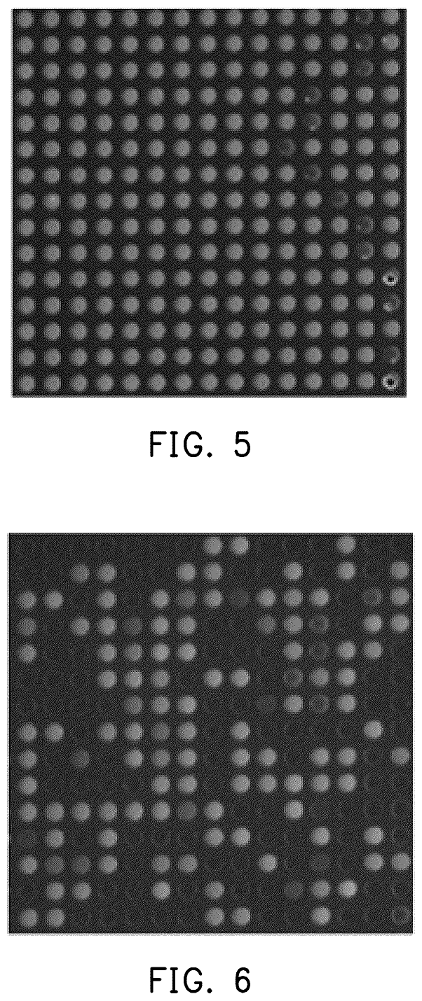

FIG. 5 is a partially enlarged view of FIG. 3, wherein some microwells are poorly dispersed due to surface defects or flow channel defects of the microwell array chip, resulting in poor dispersion of the LAMP reaction reagent sample 03. FIG. 6 is a partial fluorescence image of a microwell array chip taken using the fluorescence photography system of FIG. 1 after heating the microwell array chip (the same region as FIGS. 5) to 65° C. for 45 minutes. The excitation light source was a 470 nm LED, and the exposure time was 780 ms. The results show that there are target nucleic acid (λDNA) amplification reactions in some of the microwells, and stronger green fluorescent signals can be detected. Through digitizing nucleic acid amplification and using the Poisson distribution, the objective of absolute quantification of digital LAMP nucleic acids can be achieved. However, in order to prevent quantitative bias in the digital LAMP nucleic acid amplification reaction, the microwells with poor sample dispersion (as shown in FIG. 5 or FIG. 3) may be deducted before finding the Poisson distribution.

### Example 3

### Digital PCR of Microwell Array Chip

A microwell array chip was manufactured by injection molding and UV glue lamination, and the surface of the microwell array chip was made hydrophilic by coating or surface modification, wherein the diameter of microwell was 130 μm, the depth of microwell was 70 μm, and the pitch of microwells was 200 μm. There was a flow channel with a height of 300 μm, a width of 5 mm, and a length of 60 mm above the microwell array. Two ends of the flow channel were respectively provided with an injection hole and a ventilation hole. A PCR reaction reagent sample 04 was prepared. The PCR reaction reagent sample 04 included primers, target nucleic acid, enzyme, fluorescent indicator molecules for PCR amplification (for example, SYBR Green fluorescent dye), etc. 12 μL of the PCR reaction reagent sample 04 and 80 μL of fluorinated liquid (FC40) were sequentially injected into the flow channel of the microwell array chip from the injection hole using a micropipette. During the process, the fluorinated liquid (FC40) pushed the PCR reaction reagent sample 04 from the injection hole end toward the ventilation hole end, and the PCR reaction reagent sample 04 was dispersed in multiple microwells and was isolated by the fluorinated liquid (FC40). Subsequently, the microwell array chip was heated using a PCR temperature cycle manner to perform a digital PCR nucleic acid amplification reaction. During the dispersion process of the PCR reaction reagent sample 04, the reaction reagent sample 04 may be poorly dispersed due to surface defects or flow channel defects of the microwell array chip (such as bubbles being partially generated or some microwells being filled with less PCR reaction reagent sample 04), which causes quantitative bias in the digital PCR nucleic acid amplification reaction. Therefore, the microwells with poor sample dispersion must be deducted before finding a Poisson distribution.

Before heating the microwell array chip in the PCR temperature cycle manner, fluorescence images of the microwell array chip were taken using the fluorescence photography system of FIG. 1. The excitation light source was a 470 nm LED, and the exposure time was 1000 ms. Before a nucleic acid amplification reaction, parts of nucleic acid primers in the PCR reaction reagent sample 04 were combined with fluorescent indicator molecules for nucleic acid amplification (for example, SYBR Green fluorescent dyes). The combined fluorescent indicator molecules for nucleic acid amplification were excited using the 470 nm excitation light source to generate fluorescence. During the dispersion process of the PCR reaction reagent sample 04, the reaction reagent sample 04 may be poorly dispersed due to surface defects or flow channel defects of the microwell array chip (such as bubbles being partially generated or some microwells being filled with less PCR reaction reagent sample 04). Which microwells have bubbles or are filled with less PCR reaction reagent sample 04 may be confirmed using the fluorescence images of the microwell array chip before the PCR reaction.

After heating the microwell array chip in the PCR temperature cycle manner, fluorescence images of the microwell array chip were taken using the fluorescence photography system of FIG. 1. The excitation light source was a 470 nm LED, and the exposure time was 1500 ms. If there are target nucleic acid amplification reactions in some of the microwells, stronger green fluorescent signals can be detected. Through digitizing nucleic acid amplification and using the Poisson distribution, the objective of absolute quantification of digital PCR nucleic acids can be achieved. However, in order to prevent quantitative bias in the digital PCR nucleic acid amplification reaction, the microwells with poor sample dispersion may be deducted before finding the Poisson distribution.

In summary, the disclosure provides the confirming method for the dispersion conditions of the digital nucleic acid amplification reaction samples in the reaction areas, which may be used to confirm whether the nucleic acid samples are effectively and evenly dispersed in the microwells or the microdroplets and may be applied to digital PCR or digital LAMP. The microwells with poor sample dispersion may be deducted before finding the Poisson distribution to reduce detection errors. In addition, in the disclosure, the fluorescence is excited using the excitation light source with the first fluorescence excitation band, and the fluorescence images are detected using the detector for detecting the first fluorescence emission band, and whether the samples are effectively and evenly dispersed in the reaction areas is then determined using the fluorescence images. The first fluorescence excitation band of the excitation light source and the first fluorescence emission band of the detector before the digital nucleic acid amplification reaction are the same as the second fluorescence excitation band of the excitation light source and the second fluorescence emission band detected by the detector after the digital nucleic acid amplification reaction. Therefore, compared with the prior art, cumbersome experimental methods and additional burden can be further reduced, thereby preventing affecting the convenience and efficiency of experiments.

What is claimed is:

1. A confirming method for dispersion conditions of digital nucleic acid amplification reaction samples in reaction areas, comprising:
dispersing the nucleic acid reaction samples in the reaction areas, wherein the nucleic acid reaction samples comprise nucleic acid primers and fluorescent indicator molecules for nucleic acid amplification;
combining parts of the nucleic acid primers with the fluorescent indicator molecules for nucleic acid amplification before a digital nucleic acid amplification reaction;
exciting fluorescence using an excitation light source with a first fluorescence excitation band, and detecting a plurality of fluorescence images using a detector for detecting a first fluorescence emission band for the nucleic acid reaction samples dispersed in the reaction areas; and
determining whether the nucleic acid reaction samples are evenly distributed in the reaction areas using the fluorescence images,
wherein the first fluorescence excitation band of the excitation light source and the first fluorescence emission band of the detector before a digital nucleic acid amplification reaction are the same as a second fluorescence excitation band of the excitation light source and a second fluorescence emission band detected by the detector after the digital nucleic acid amplification reaction.

2. The confirming method for the dispersion conditions of the digital nucleic acid amplification reaction samples in the reaction areas according to claim 1, wherein the digital nucleic acid amplification reaction comprises digital polymerase chain reaction (PCR) or digital loop-mediated isothermal amplification (LAMP).

3. The confirming method for the dispersion conditions of the digital nucleic acid amplification reaction samples in the reaction areas according to claim 1, wherein the reaction areas comprise a plurality of microwells or a plurality of microdroplets.

4. The confirming method for the dispersion conditions of the digital nucleic acid amplification reaction samples in the reaction areas according to claim 1, wherein a concentration of primer is 0.2 μM to 1.6 μM, and a concentration of fluorescent indicator molecules for nucleic acid amplification is 0.5 μM to 1 μM.

5. The confirming method for the dispersion conditions of the digital nucleic acid amplification reaction samples in the reaction areas according to claim 1, wherein the fluorescent indicator molecules for nucleic acid amplification comprise fluorescent dyes, and the fluorescent dyes comprise SYBR Green, EvaGreen, or SYTO.

6. The confirming method for the dispersion conditions of the digital nucleic acid amplification reaction samples in the reaction areas according to claim 1, wherein 5% to 95% of the nucleic acid primers are combined with the fluorescent indicator molecules for nucleic acid amplification.

7. The confirming method for the dispersion conditions of the digital nucleic acid amplification reaction samples in the reaction areas according to claim 1, wherein passband of the first fluorescence excitation band and the second fluorescence excitation band is from 446 nm to 486 nm, and passband of the first fluorescence emission band and the second fluorescence emission band is from 506 nm to 534 nm.

8. The confirming method for the dispersion conditions of the digital nucleic acid amplification reaction samples in the reaction areas according to claim 1, wherein a method for determining whether the nucleic acid reaction samples are evenly dispersed in the reaction areas using the fluorescence images comprises analyzing and comparing fluorescence intensities of the fluorescence images.
